(19) United States
(12) Patent Application Publication
(10) Pub. No.: US 2008/0050432 A1
(43) Pub. Date: Feb. 28, 2008

(54) Combined Pharmaceutical Formulation with Controlled-Release Comprising Dihydropyridine Calcium Channel Blockers and HMG-COA Reductase Inhibitors

(76) Inventors: Sung Soo Jun, Gyeonggi-do (KR); Young Gwan Jo, Daejeon (KR); Ja-Seong Koo, Daejeon (KR); Jin Wook Kim, Daejeon (KR); Ju-Bin Yim, Daejeon (KR)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/696,942

(22) Filed: Apr. 5, 2007

(30) Foreign Application Priority Data
Aug. 24, 2006 (KR) 10-2006-0080694

(51) Int. Cl. A61K 9/56; A61K 9/36; A61P 9/10; A61K 31/44; A61P 9/12; A61K 9/24; A61K 9/30

(52) U.S. Cl. 424/472; 424/475; 424/476; 424/480; 514/356; 514/460; 424/468; 424/472; 514/277

(57) ABSTRACT

The present invention relates to a combined pharmaceutical formulation, which is such designed that the release of each ingredient may be controlled to a predetermined release rate by applying the principle of the so-called chronotherapy, where drugs are administered in such a way that the activities of the drugs are expressed at intervals. The formulation of the present invention comprises statin-based lipid-lowering agent and dihydropyridine-based calcium channel blocker that affects cytochrome P450 enzyme as active ingredients, and is such constituted that the release rates of the aforementioned ingredients are different, thus preventing antagonism and side effects, while maintaining the synergistic effect, which leads to the convenience in medication.

### TECHNICAL FIELD

The present invention relates to a combined pharmeceutical formulation with controlled-release comprising a dihydropyridine-based calcium channel blocker and a statin-based lipid-lowering agent. The formulation of the present invention is designed in such a manner that the release of each ingredient may be controlled to a predetermined rate by applying the principle of the so-called chronotherapy, where drugs are administered so that the activities of the drugs are expressed at certain intervals for better therapeutical effect and less side effect.

### RELATED PRIOR ART

Arteriosclerosis and hypertension aggravate symptoms of each other in a vicious circle, and the aggravation may be prevented only by treatment of both arteriosclerosis and hypertension at the same time in patients suffering from hyperlipidemia and hypertension [Hypertens Res 2001; 24: 3-11, Hypertens Res 2003; 26:1-36, Hypertens Res 2003; 26: 979-990].

Thus, there have been clinical results reported about the synergistic effect of the administration of a lipid-lowering agent and a calcium channel blocker with a lipid-lowering effect in combination with a statin-based cholesterol synthesis inhibitor. According to Kramsch et al., a combination of amlodipine and a lipid-lowering agent shows a better therapeutical effect for atherosclerosis [Journal of Human Hypertension (1995) (Suppl. 1), 53-59]. Jukema et al. also proved the synergistic effect when a calcium channel blocker and a lipid-lowering agent were combined [Circulation, 1995 (Suppl. 1), 1-197].

A combination of amlodipine as a representative calcium channel blocker, particularly a dihydropyridine-based calcium channel blocker along with simvastatin as a statin-based lipid-lowering agent is a most widely used combined prescription.

It is well known that amlodipine serves as a medication for stenocardia as well as hypertension. Simvastatin is also well known to have a lipid-lowering activity and an anti-cancer activity on the wall of blood vessel. Stability of these drugs are also well known such and are available without prescription in a pharmacy in Great Britain [Cardiology 1992; 80 (Suppl 1): S31-S36, J Cardiovasc Pharmacol 1988; 12 (Suppl 7): S110-S113, Lancet 2000; 356: 359-365, Hypertens Res 2002; 25: 717-725, Hypertens Res 2002; 25: 329-333].

Meanwhile, amlodipine serves as an anti-cancer medicine and also increases a lipid-lowering activity of simvastatin through a synergistical activity with a lipid-lowering agent. Simvastatin serves as a lipid-lowering agent and also has an activity of decreasing blood pressure through a synergistic activity with amlodipine. The aforementioned two drugs are both administered once daily, and the medication for both drugs is preferred to be administered with dinner.

A representative statin-based lipid-lowering agent, simvastatin has the following information.

That is, it is well known that HMG-COA reductase inhibitor, a statin-based lipid-lowering agent is the first option for prevention and treatment of heart diseases due to coronary arteriosclerosis such as stenocardia or myocardial infarction [Lancet 1995; 346: 750-753, Am J Cardiol 1998; 82: 57T-59T, Am J Cardiol 1995; 76: 107C-112C, Hypertens Res 2003; 26: 699-704, Hypertens Res 2003; 26: 273-280.] Br Med Bull 2001; 59: 3-16, Am J Med 1998; 104 (Suppl 1): 6S-8S, Clin Pharmacokinet 2002; 41: 343-370.].

Moreover, simvastatin is most frequently prescribed among statin-based lipid-lowering agents, and has been well known to decrease the rate of coronary arteriosclerosis and the death rate through a large-scale clinical test [Lancet 1994; 344: 1383-1389.].

The aforementioned activities are due to the fact that simvastatin strongly inhibits HMG-CoA reductase, which plays a key role in the synthesis of cholesterol in liver and also inhibits an inflammation-inducing factor [“Scandinavian Simvastatin Survival Study” published in the Lancet, 1994, 344, 1383-89].

Patients suffering from atherosclerosis or diabetes show abnormal NO synthase (eNOs) in blood vessel wall, and the blood pressure increases due to the decrease in NO generation. Statin-based lipid-lowering agent including simvastatin increases the e-NOS to a normal level, which is also an effect of a combined prescription where a lipid-lowering activity helps an anti-cancer activity [Am J Physiol Renal Physiol Vol 281 Issue 5: F802-F809, 2001].

Simvastatin is an inactivated lactone, which enters liver first and is then transformed into an activated form, simvastatin acid, with lipid-lowering activity. The remaining simvastatin is also metabolized through various steps by cytochrome P450 3A4 in liver, and some of metabolites shows a strong lipid-lowering activity.

The simvastatin and simvastatin acid are metabolized by cytochrome P450 3A4, functions in liver and excreted from liver [Drug Metab Dispos 1990; 18: 138-145, Drug Metab Dispos 1990; 18: 476-483, Drug Metab Dispos 1997; 25: 1191-1199.].

Thus, when used in combination with a drug inhibiting cytochrome P-450 3A4 enzyme, simvastatin is subject to less metabolism in liver and the plasma concentration of simvastatin is increased, which may lead to serious side effects such as muscular domyolysis [Clin Pharmacol Ther 1998; 63: 332-341, Clin Pharmacol Ther 1998; 64: 177-182, Physicians Desk Reference 2006 (Zocor), J Pliarmacol Exp Ther 1997; 282: 294-300, Pharmacol Exp Ther 1999; 290: 1116-1125, Life Sci 2004; 76: 281-292.].

Therefore, a very specially designed administration should be employed in a combined prescription with drugs such as amlodipine that inhibit cytochrome P450 3A4 enzyme essential in metabolism in statin-based drug such as simvastatin. Statin-based drugs has been recommended to be administered early in the evening because lipid synthesis in liver is very active [Arterioscler Thromb 11: 816-826, Clinic Oharmacol Ther 40: 338-343.].

A representative calcium channel blocker, amlodipine has the following information.

Calcium channel blocker is an anti-cancer medicine that is most frequently prescribed in combination with simvastatin. Particularly, amlodipine is the most widely prescribed in the world as an anti-cancer medicine and a medicine for stenocardia [Cardiology 1992; 80 (Suppl 1): S31-S36, J Cardiovasc Pharmacol 1988; 12 (Suppl 7): S110-S113, Lancet 2000; 356: 359-365, Hypertens Res 2002; 25: 717-725, Hypertens Res 2002; 25: 329-333.].

Amlodipine, which is used in the present invention in combination with statin-based lipid-lowering agent represented by simvastatin, is a controlled-release drug belonging to dihydropyridine-based calcium channel blocker [Clin Pharmacokinet 1992; 22: 22-31, Am Heart J 1989; 118: 1100-1103, Hypertens Res 2003; 26: 201-208.].

Amlodipine, which has a chemical name of 3-ethyl-5-methyl-2-(2-amino ethoxymethyl)-4-(2-chlorophenyl)-1,4-dihydro-6-methyl-3,5-pyridinedicarboxylate, is a very useful calcium channel blockers that has a half-life of 30-50 hours and shows an activity for a relatively long period of time [European patent publication No. 89,167 and U.S. Pat. No. 4,572,909]. Amlodipine is also a medicine for hypertension, which prevents calcium from being flowed into a vascular smooth muscle and induces the expansion of pheriphery artery, thus lowering blood pressure. Amlodipine is a useful drug for stenocardia due to spasmodic contraction of vascular wall. When orally administered in the form of a single pill, amlodipine is absorbed in small intestine. Then, more than 40% is resolved in liver and only the remaining 60% is present in blood, thus sufficiently exerting a pressure-lowering activity.

Amlodipine continues the activity for 24 hours, and shows strongest activity of lowering blood pressure during the time from morning to noon when administered in the evening of the previous day.

From a pathophysiological point of view, the pressure increase in the day time is caused by the spasm of vascular wall due to stress stimulus. Amlodipine functions an activity of relaxing the spasmodic contraction of vascular wall, and shows a strong activity of lowering blood pressure in the day time. Thus, amlodipine administered during the evening reaches the maximum plasma concentration in the morning and shows the strongest activity in the day time [Hypertens 10(Supp; 4): S136, Clin Invest 72: 864-869].

In the presence of cytochrome P450 3A4 enzyme, some of amlodipine is oxidized by the enzyme and metabolized into an inactive material. However, amlodipine immediately shows an activity of inhibiting the generation of cytochrome P450 3A4 enzyme.

Due the aforementioned nature, amlodipine should be administered at certain intervals when administered in combination with statin-based lipid-lowering agent such as simvastatin that needs cytochrome P450 3A4 enzyme [Med Chem 1991; 34: 1838-1844, Eur J Clin Pharmacol 2000; 55: 843-852.].

Because the combined prescription of amlodipine and simvastatin has the problems mentioned below, a combined pharmaceutical formulation of amlodipine and simvastatin has not been approved, and only a combined prescription of single preparations has been performed without appropriate medication instruction.

That is, there are very few who give a patient an instruction to take two drugs in the evening at intervals, and the majority of patients do not know when to take the two drugs.

However, the simultaneous administration of the aforementioned drugs may increase the plasma concentration of simvastatin by 30% thus generating side effects. It is also difficult to expect an effective activity of lowering blood pressure and lipid from the combined administration of the two drugs.

Shinichiro Nishio et al. reported the result of experiments comparing between two groups of patients suffering from hyperlipidemia. One group was administered with amlodipine single pill and simvastatin single pill at the same time, and the other group was administered with simvastatin single pill only [Hypertens Res, 2005, Vol. 28, No. 3].

According to the experiments, the simultaneous administration of simvastatin and amlodipine inhibits cytochrome P450 3A4 enzyme due to amlodipine and increases the plasma concentration of simvastatin by 30% leading to the possibility of side effects [Hypertension Research Vol. 28 (2005), No. 3 March 223-227].

TABLE1

| Daily dosage | Cmax (ng/ml) | AUC (ng/ml) |
|---|---|---|
| Simvastatin 5 mg | 9.6 ± 3.7 | 34.3 ± 16.5 |
| Simvastatin 5 mg + | 13.7 ± 4.7 | 43.9 ± 16.6 |
| Amlodipine 5 mg | | |

Hypertension Research Vol.25 (2005). No. 3 March 223–227

As shown in TABLE 1, as compared to the administration of simvastatin only, the combined prescription was higher by 30% in the plasma concentration of the lipid-lowering ingredient. Nevertheless, the lipid-lowering activity was not increased. At a higher plasma concentration than a certain level, simvastatin decreases in an activity of inhibiting biosynthesis of cholesterol, and is likely to incur serious side effects such as muscular domyolysis.

Thus, there are needs for developing a novel medication method or a novel pharmaceutical formulation that may prevent the drawback of the combined prescription of single pills, i.e. the antagonism between the drugs.

Therefore, such a specially combined pharmeceutical formulation that may overcome the antagonism between drugs has been developed in the present invention by employing a pharmaceutical concept that the drugs are dissolved at prescheduled intervals.

The present inventors have exerted extensive researches to develop a way to solve the aforementioned problems and increase the therapeutical effect of the combined prescription, which is clinically inevitable, while reducing the side effects.

As a result, the present invention has been completed based on the finding that the plasma concentration of a statin-based lipid-lowering agent may be deterred from being increased to a level higher than a certain level and the side effect may be prevented by applying a prescheduled interval between the absorption times of a statin-based lipid-lowering agent such as simvastatin and a dihydropyridine-based calcium channel blocker such as amlodipine within the gastrointestinal tract.

In the case of a complex system of the present invention, statin-based lipid-lowering agent is absorbed first immediately after the administration of the formulation and transformed into an activated form, and inactivated metabolites are totally metabolized by cytochrome P450 3A4 enzyme and excreted. After enough time from when simvastatin is affected by cytochrome P450 3A4 enzyme or the formulation is administered, a dihydropyridine-based calcium channel blocker such as amlodipine is absorbed in the gastrointestinal tract and simvastatin may not be affected by the inhibition of amlodipine by cytochrome P450 3A4.

Considering that simvastatin, a first-pass metabolite that enters liver after being absorbed in small intestine, shows an inhibitory activity against the lipid synthesis in liver, the present invention improves a controlled-release system in such a way that simvastatin may not be released into a higher plasma concentration than a certain level by allowing simvastatin to stay in iver and show its activity.

That is, the present invention is related controls the controlled-release ingredients by constituting the formulation comprising a controlled-release part containing a dihydropyridine-based calcium channel blocker as an active ingredient and an immediate-release part containing statin-based lipid-lowering agent as an active ingredient, thus enabling the dihydropyridine-based calcium channel blocker to be dissolved or absorbed in the small intestine 3-4 hours later than the statin-based lipid-lowering agent.

As compared to the single pills of a dihydropyridine-based calcium channel blocker and a statin-based lipid-lowering agent, the combined pharmaceutical formulation of the present invention comprising a dihydropyridine-based calcium channel blocker and a statin-based lipid-lowering agent shows a by far superior pharmacokinetics of the statin-based lipid-lowering agent.

According to the present invention, a single formulation for oral administration provides a synergistic effect of a combined administration of the dihydropyridine-based calcium channel blocker and the statin-based lipid-lowering agent, maximizing the pharmaceutical activity of each active ingredient by inhibiting the competitive antagonism of drugs according to absorption, metabolism and mechanism in a body with the lapse of time through the control of release while minimizing side effects, and increases the patient compliance due to the convenience in taking medicine (i.e. one pill daily in the evening).

In principle, a drug should not be administered in combination with another drug unless the advantages due to the combined administration exceed the dangers to be resulted therefrom. Considering the great synergistic clinical effects of a combined administration of a dihydropyridine-based calcium channel blocker and a statin-based lipid-lowering agent, particularly amlodipine and simvastatin, the present invention maintains the synergistic effects and also eliminates the side effects by resolving the side effect of simvastatin, e.g. myopathy due to the combined administration despite the inhibitory effect of amlodipine against cytochrome P450 3A4.

Therefore, the present invention aims to provide a combined pharmeceutical formulation comprising a controlled-release dihydropyridine-based calcium channel blocker and a statin-based lipid-lowering agent.

### BRIEF DESCRIPTION of DRAWINGS

FIG. 1 shows a graph comparing dissolution rates between the amlodipine/simvastatin two-phase matrix tablets prepared in Example 1 and the control drugs (Zocor: simvastatin single pill, Norvasc: amlodipine single pill).

FIG. 2 shows a graph comparing dissolution rates between the amlodipine/simvastatin combined pharmeceutical formulation prepared in Examples 5 and 6 and the control drugs (Zocor: simvastatin single pill, Norvasc: amlodipine single pill).

FIG. 3 shows a graph comparing dissolution rates between the amlodipine/lovastatin combined pharmeceutical formulation prepared in Example 11 and the control drugs (Mevacor: lovastatin single pill, Norvasc: amlodipine single pill).

FIG. 4 shows a graph comparing dissolution rates between the amlodipine/atrovastatin combined pharmeceutical formulation prepared in Example 13 and the control drugs (Lipitor: atrovastatin single pill, Norvasc: amlodipine single pill).

FIG. 5 shows a graph comparing dissolution rates between the lercanidipine/simvastatin combined pharmeceutical formulation prepared in Example 15 and the control drugs (Zocor: simvastatin single pill, Zanidip: lercanidipine single pill).

FIG. 6 shows a graph comparing dissolution rates between the lacidipine/simvastatin combined pharmeceutical formulation prepared in Example 17 and the control drugs (Zocor: simvastatin single pill, Vaxar: lacidipine single pill).

### DETAILED DESCRIPTION OF INVENTION

The present invention relates to a combined pharmeceutical formulation comprising a dihydropyridine-based calcium channel blocker and a statin-based lipid-lowering agent as active ingredients and a pharmaceutically acceptable carrier, the combined pharmaceutical formulation including a controlled-release part comprising the dihydropyridine-based calcium channel blocker as an active ingredient and an immediate-release part comprising the statin-based lipid-lowering agent as an active ingredient.

Hereunder is provided a detailed description of the present invention.

The present invention relates to a combined pharmeceutical formulation, which is such designed that the release of each ingredient may be controlled to a predetermined rate by applying the principle of the so-called chronotherapy, where drugs are administered so that the activities of the drugs are expressed at intervals. The formulation of the present invention comprises statin-based lipid-lowering agent and dihydropyridine-based calcium channel blocker that affects cytochrome P450 enzyme as active ingredients, and is such constituted that the release rates of the aforementioned ingredients are different, thus preventing antagonism and side effects, while maintaining the synergistic effect, which provides convenience in medication.

Hereunder is provided a detailed description of the combined pharmaceutical formulation according to the present invention, which comprises a dihydropyridine-based calcium channel blocker and a statin-based lipid-lowering agent.

The combined pharmaceutical formulation of the present invention comprises a dihydropyridine-based calcium channel blocker and a statin-based lipid-lowering agent as active ingredients. A compound that may be inhibited by cytochrome P450 enzymes may be selected as the dihydropyridine-based calcium channel blocker. Examples of the dihydropyridine-based calcium channel blocker include without limitation amlodipine, lercanidipine, lacidipine and a pharmaceutically acceptable salt thereof. Preferably, amlodipine or a pharmaceutically acceptable salt thereof or an isomer thereof, specifically amlodipine maleate and amlodipine besylate, may be used as the dihydropyridine-based calcium channel blocker. Preferable daily dosage of the dihydropyridine-based calcium channel blocker is 1-20 mg (for an adult weighing 65-75 kg), and it may be contained in an amount of 1-20 mg, preferably 5-10 mg in a tablet of the present invention.

As the dihydropyridine-based calcium channel blocker having an efficacy of lowering blood pressure, the present application specifically describes amlodipine. However, the present invention shall not be limited to amlodipine.

Simvastatin, lovastatin and atrovastatin may be used as the statin-based lipid-lowering agents. The daily dosage of the statin-based lipid-lowering agent for an adult is 5-80 mg, and it may be contained in an amount of 5-80 mg, preferably 10-40 mg in a tablet of the present invention.

Representative example of the statin-based lipid-lowering agent is simvastatin, and the present invention describes simvastatin as a specific example. However, the present invention is limited to simvastatin in no way. Although simvastatin is inactive material, it may be changed into an active simvastatin acid by esterase, and further changed into an activated form by cytochrome P450 3A4 in liver, thereby exerting a lipid-inhibiting activity.

Meanwhile, amlodipine inhibits the activity of the cytochrome P450 3A4. Therefore, when amlodipine and simvastatin are administered at the same time, amlodipine that is rapidly absorbed into the small intestine reaches liver earlier than simvastatin and thereby inhibits the induction of cytochrome P450 3A4. Hence, a considerable portion of the simvastatin that reaches liver later or at the same time is not subject to the metabolic activity of cytochrome P450 3A4 and more than 30% of the simvastatin may leak into blood, show delayed excretion or may be accumulated. As a result, simvastatin or simvastatin acid that is not metabolized by the cytochrome P450 3A4 moves into the blood and may cause a muscular disorder such as muscular domyolysis due to the elevated plasma concentration of simvastatin or simvastatin acid.

As a way to solve the aforementioned problem and prevent amlodipine from inhibiting the absorption of simvastatin into liver, the present invention constitutes an immediate-release part that releases simvastatin first and causes simvastatin to be absorbed into the small intestine earlier, while constituting a controlled-release part that causes amlodipine to be absorbed into liver 3-4 hours later than simvastatin.

The novel composition of the present invention comprises a controlled-release composition containing amlodipine, a pharmaceutically acceptable salt thereof and desired excipients and an immediate-release composition containing simvastatin and desired excipients, which is physically separated or partitioned so that two different drugs show different release rates. Moreover, the immediate-release part and the controlled-release part may be formulated into various forms.

That is, the novel pharmaceutical composition may be coated according to a conventional method by using a release controlling material selected among the group comprising the controlled-release part, and thus obtained coated particles or granules and multi-component particles or granules of an immediate-release simvastatin composition may be compressed into a tablet or filled in a capsule.

The controlled-release part of the present invention contains dihydropyridine-based calcium channel blocker such as amlodipine, and an enteric polymer, a water-insoluble polymer, a hydrophobic compound, a hydrophilic nonpolymeric compound and a hydrophilic polymer as a release controlling material thereof. The release controlling material in the controlled-release part may be contained in an amount of 10-500 weight parts relative to 100 weight parts of the dihydropyridine-based calcium channel blocker. If the amount is below the above range, the release control may not be sufficient. If the amount is above the range, the release of drug is retarded and statistically significant clinical effect may not obtained.

Examples of the enteric polymer include but are not limited to poly(vinylacetate phthalate-co-methacrylic acid) copolymer, hydroxypropylmethyl cellulose phthalate, shellac, cellulose acetate phthalate, cellulose propionate phthalate, Eudragit L, Eudragit S and a mixture thereof may be used.

Examples of the water-insoluble polymer include but are not limited to a pharmaceutically acceptable poly(vinylacetate-co-methacrylate) copolymer such as poly(ethylacrylate-co-methylmethacrylate) copolymer, poly(ethylacrylate-methyl methacrylate-trimethyl aminoethyl methacrylate) copolymer, ethyl cellulose, cellulose acetate and a mixture thereof may be used.

Examples of the hydrophobic organic compound include but are not limited to a fatty acid and a fatty acid ester, a fatty acid alcohol, a wax, an inorganic material and a mixture thereof may be used. Specifically, examples of the fatty acid and fatty acid esters include but are not limited to glyceryl palmitostearate, glyceryl stearate, glyceryl behenate, cetyl palmitate, glyceryl mono oleate, stearic acid and a mixture thereof may be used; examples of the fatty acid alcohol include but are not limited to cetostearyl alcohol, cetyl alcohol, stearyl alcohol and a mixture thereof; examples of the wax include but are not limited to Carnauba wax, beeswax, noncrystalline wax and a mixture thereof; and the examples of the inorganic material include but are not limited to talc, precipitated calcium carbonate, dibasic calcium phosphate, zinc oxide, titanium oxide, kaolin, bentonite, montmorillonite, veegum and a mixture thereof.

Examples of the hydrophilic polymer include but are not limited to a saccharide, a cellulose derivative, a gum, a protein, a polyvinyl derivative, a polymethacrylate copolymer, a polyethylene derivative, a carboxyvinyl polymer and a mixture thereof. Specifically, examples of the saccharide include but are not limited to dextrin, polydextrin, dextran, pectin and pectin derivative, alginate, poly(galacturonic acid), xylan, arabinoxylan, arabinogalactan, starch, hydroxypropyl starch, amylase, amylopectin and a mixture thereof; examples of the cellulose derivative include but are not limited to hydroxypropylmethyl cellulose, hydroxypropyl cellulose, hydroxymethyl cellulose, hydroxyethyl cellulose, methyl cellulose, carboxymethyl cellulose sodium, hydroxypropyl methyl cellulose acetate succinate, hydroxyethylmethyl cellulose and a mixture thereof; examples of the gums include but are not limited to guar gum, locust bean gum, tragacantha, carrageenan, gum acasia, gum arabic, gellan gum, xanthan gum and a mixture thereof; examples of the proteins include but are not limited to gelatin, casein, zein and a mixture thereof; examples of the polyvinyl derivative include but are not limited to polyvinyl alcohol, polyvinyl pyrrolidone, polyvinylacetal diethylaminoacetate and a mixture thereof; examples of the polymethacrylate copolymer include but are not limited to poly(butyl methacrylate, (2-dimethylaminoethyl)methacrylate, methylmethacrylate) copolymer, poly(methacrylic acid, methylmethacrylate) copolymer, poly(methacrylic acid, ethylacrylate) copolymer and a mixture thereof; examples of the polyethylene derivative include but are not limited to polyethylene glycol, polyethylene oxide and a mixture thereof; and examples of the carboxyvinylpolymer include but are not limited to carbomer.

The controlled-release part of the present invention consists of discontinuous phases of particles or granules prepared by mixing, granulating or coating a dihydropyridine-based calcium channel blocker, a release controlling material and commonly used pharmaceutical excipients.

The immediate-release part of the present invention may be prepared into particles or granules by performing normal processes for manufacture of oral solid forms such as mixing, combining, drying and granulation using statin-based lipid-lowering agent such as simvastatin as an active ingredient and a pharmaceutically acceptable excipient. If the fluidity of simvastatin mixture is good enough for direct compression, the mixture may be mixed to provide composition, while if the fluidity is not good, a composition may be prepared by pressurization, granulation and grinding, thus enabling to prepare a continuous phase comprising an immediate-release part.

A formulation for oral administration comprising a controlled-release part and an immediate-release part matrix in two phases by post-mixing a composition contained in the controlled-release part and the immediate-release part with pharmaceutically acceptable additives for compression or by filling the composition into a capsule.

For example, the formulation according to the present invention may be prepared into two-phase matrix partitioned in a single pill by granule phase, multi-layered tablet, inner core tablet or a capsule filled with granules of a controlled-release part and an immediate-release part. Moreover, the formulation may also be prepared into a tablet comprising a controlled-release inner core tablet containing amlodipine and an immediate-release double inner core tablet containing simvastatin.

However, the formulation according to the present invention is not limited to a single two-phase matrix tablet where a discontinuous phase of a controlled-release amlodipine exists in a continuous phase of an immediate-release simvastatin.

That is, a table for oral administration having layers for an immediate-release or a controlled-release by mixing granules contained in the controlled-release part and the immediate-release part with pharmaceutically acceptable excipients, followed by compression into a double-layered or a triple-layered tablet where layers are parallel to each other using a compressor for the production of a multi-layered tablet. Moreover, a tablet for oral administration having a structure of a controlled-release layer as an inner core and an immediate-release layer encompassing the inner core by mixing and compressing the granulates contained in the controlled-release part with pharmaceutically acceptable excipient to provide an inner core tablet and by mixing and compressing the granulates contained in the immediate-release part with a pharmaceutically acceptable excipient. Furthermore, a capsule formulation for oral administration, where the two-phase release-control is possible, can be obtained by mixing granulates contained in the controlled-release part and the immediate-release part with a pharmaceutically acceptable excipient and filling the mixture in a capsule.

Besides the active ingredient and the release controlling material, the formulation of the present invention may further comprise such amounts of other additional ingredients that the effect of the present invention may not be damaged. Examples of a pharmaceutically acceptable diluent as the aforementioned additives include without limitation starch, microcrystalline cellulose, lactose, glucose, mannitol, alginate, salt of alkaline earth metal, clay, polyethylene glycol and dicalcium phosphate. Examples of a binding agent as the aforementioned additives include without limitation starch, microcrystalline cellulose, highly-dispersed silica, mannitol, lactose, polyethylene glycol, polyvinylpyrrolidone, hydroxypropyl methyl cellulose, hydroxypropyl cellulose, natural gum, synthetic gum, copovidone and gelatin. Examples of a disintegrant as the aforementioned additives include without limitation starch or denatured starch such as sodium starch glycolate, corn starch, potato starch and pre-gelatinated starch; clay such as bentonite, montmorillonite and veegum; celluloses such as microcrystalline cellulose, hydroxypropyl cellulose and carboxymethyl cellulose; aligns such as sodium alginate or alginic acid; crosslinked celluloses such as croscarmellose sodium; gums such as guar gum and xanthan gum; a crosslinked polymer such as crospovidone; and effervescent formulation such as sodium bicarbonate and citric acid. Examples of an eluent as the aforementioned additives include without limitation talc, magnesium stearate and alkaline earth metal stearate type calcium, zinc, etc, lauryl sulfate, hydrogenated vegetable oil, sodium benzoate, sodium stearyl fumarate, glyceryl monostearate and polyethylene glycol 4000. Other pharmaceutically acceptable additives such as coloring agents or perfumery may be used.

Although noncrystalline cellulose, sodium starch glyconate, colloidal silicon dioxide, magnesium stearate, etc are used in Examples herein as the additives, the present invention is limited to the aforementioned additives in no way, and the usage of the additives may be easily determined by one skilled in the art.

The formulation may optionally comprise a coating layer on the surface of the tablet. That is, the amlodipine/simvastatin combined pharmaceutical formulation of the present invention may be formulated into an uncoated form or a coated tablet for better stability of active ingredients.

The coating layer may be formed on the surface of tablet using the aforementioned ingredients by conventional methods such as a fluidized-bed coating method and, preferably, a fan coating method.

The coating layer may be prepared using a film former, a film-forming adjuvant or a mixture thereof. In particular, the coating layer may be prepared using cellulose derivative, saccharide derivative, polyvinyl derivative, waxes, lipids, gelatin and a mixture as a film former; polyethylene glycol, ethyl cellulose, glycerides, titanium oxide, diethyl phthalate and a mixture thereof as a film-forming adjuvant.

The coating layer is preferred to be contained in an amount of 0.5-15 wt % of total weight of the coated tablet.

The combined pharmeceutical formulation of the present invention is prepared into a single combined pharmeceutical formulation containing amlodipine and simvastatin as active ingredients, and may be administered once daily in the evening. Hence, as compared to separate formulations to be administered simultaneously, the combined pharmeceutical formulation of the present invention has advantages of easily medication instruction, lowered side effect due to the antagonism between drugs and superior activity of controlling blood tension and lipid.

When administered orally, the combined pharmeceutical formulation of the present invention shows an immediate-release of simvastatin and releases more than 80% of initial amount of simvastatin with one hour, and shows a controlled-release of amlodipine and releases less than 50% of initial amount of amlodipine within one hour. It is preferable that more than 90% of initial amount of simvastatin and at least 40% of initial amount of amlodipine are released within one hour.

The combined pharmeceutical formulation of the present invention may be used for prevention and treatment of hypertension, stenocardia, atherosclerosis and arteriosclerosis, which may result in apoplexy, heart attack and kidney transplantation.

### EXAMPLES

The present invention is described more specifically by the following Examples. Examples herein are meant only to illustrate the present invention, but in no way to limit the claimed invention.

### Example 1

### Preparation of Amlodipine-Simvastatin Two-Phase Matrix Tablets

### 1) Preparation of Amlodipine Controlled-Release Granule

Predetermined amounts of amlodipine and noncrystalline cellulose as shown in TABLE 2 were sieved with a 35 mesh sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules. After drying, the granules were coated by spraying a 5 wt % solution of hydroxypropylmethyl cellulose phthalate in a 1:1 mixture of ethanol and methylene chloride.

### 2) Preparation of Simvastatin Granule

Predetermined amounts of simvastatin, noncrystalline cellulose and mannitol as shown in TABLE 2 were sieved with a 35 mesh sieve and mixed using a high-speed mixer. A binder solution prepared by dissolving hydroxypropyl cellulose and citric acid in water, and combined with the mixture of the main ingredients. Thus obtained mixture was granulated using an oscillator with a 20 mesh sieve, dried at 60° C. using a hot-water drier, ground with a 20 mesh sieve, and mixed with butylhydroxyaniline.

### 3) Post-Mixing, Compression and Coating

The obtained composition was mixed using a double cone mixer, added with sodium starch glyconate and colloidal silicon dioxide, and mixed with magnesium stearate using a high-speed mixer.

The final composition was compressed using a rotary compressor (MRC-33: Sejong) at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing two-phase matrix tablets.

### Example 2

### Preparation of Amlodipine-Simvastatin Two-Phase Matrix Tablets

### 1) Preparation of Amlodipine Controlled-Release Granule

Predetermined amounts of amlodipine and noncrystalline cellulose as shown in TABLE 2 were sieved with a 35 mesh sieve and mixed. The mixture was mixed using Kollicoat SR30D in a high-speed mixer. Thus obtained mixture was granulated using oscillator with a 20 mesh sieve, dried at 60° C. using a hot-water drier and sized with a 20 mesh sieve.

### 2) Preparation of Simvastatin Granule

Predetermined amounts of simvastatin, noncrystalline cellulose and mannitol as shown in TABLE 2 were sieved with a 35 mesh sieve and mixed using a high-speed mixer. A binder solution prepared by dissolving hydroxypropyl cellulose and citric acid in water, and combining with the mixture of the main ingredients. Thus obtained mixture was combined, granulated using an oscillator with a 20 mesh sieve, dried at 60° C. using a hot-water drier, ground with a 20 mesh sieve, and mixed with butylhydroxyaniline.

### 3) Post-Mixing, Compression and Coating

The obtained composition was mixed using a double cone mixer, added with sodium starch glyconate and colloidal silicon dioxide, and mixed with magnesium stearate using a high-speed mixer.

The final composition was compressed using a rotary compressor (MRC-33: Sejong) at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing two-phase matrix tablets.

### Example 3

### Preparation of Amlodipine-Simvastatin Two-Phase Matrix Tablets

### 1) Preparation of Amlodipine Controlled-Release Granule

Predetermined amounts of amlodipine and noncrystalline cellulose as shown in TABLE 2 were sieved with a 35 mesh sieve, and mixed using a double cone mixer. The mixture was placed into a fluidized-bed granulator (GPCG 1: Glatt), and sprayed with a binder solution (an aqueous solution of hydroxypropylmethyl cellulose) to prepare granules. After the granules were dried, they were coated by spraying a 5 wt % solution of Eudragit RS PO in a 1:1 mixture of ethanol and methylene chloride.

### 2) Preparation of Simvastatin Granule

Predetermined amounts of simvastatin, noncrystalline cellulose and mannitol as shown in TABLE 2 were sieved with a 35 mesh sieve and mixed using a high-speed mixer. A binder solution prepared by dissolving hydroxypropyl cellulose and citric acid in water, and combining with the mixture of the main ingredients. Thus obtained mixture was granulated using an oscillator with a 20 mesh sieve, dried at 60° C. using a hot-water drier, ground with a 20 mesh sieve, and mixed with butylhydroxyaniline.

### 3) Post-Mixing, Compression and Coating

The obtained composition was mixed using a double cone mixer, added with sodium starch glyconate and colloidal silicon dioxide, and mixed with magnesium stearate using a high-speed mixer.

The final composition was compressed using a rotary compressor (MRC-33: Sejong) at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing two-phase matrix tablets.

### Example 4

### Preparation of Amlodipine-Simvastatin Multi-Layered Tablets

### 1) Preparation of Amlodipine Controlled-Release Layer

Predetermined amounts of amlodipine and noncrystalline cellulose as shown in TABLE 2 were sieved with a 35 mesh sieve, and mixed using a double cone mixer. The mixture was sprayed with a binder solution (an aqueous solution of hydroxypropylmethyl cellulose) to prepare granules. After the granules were dried, they were coated by spraying a 5 wt % solution of hydroxypropylmethyl cellulose phthalate in a 1:1 mixture of ethanol and methylene chloride. The coated granules were mixed with magnesium stearate using a double cone mixer.

### 2) Preparation of Simvastatin Layer

Predetermined amounts of simvastatin, noncrystalline cellulose and mannitol as shown in TABLE 2 were sieved with a 35 mesh sieve and mixed using a high-speed mixer. A binder solution prepared by dissolving hydroxypropyl cellulose and citric acid in water, and combining with the mixture of the main ingredients. Thus obtained mixture was combined, granulated using an oscillator with a 20 mesh sieve, dried at 60° C. using a hot-water drier and ground with a 20 mesh sieve. The granules were mixed with butylhydroxyaniline, sodium starch glyconate and colloidal silicon dioxide, and finally mixed with magnesium stearate using a double cone mixer.

### 3) Compression and Coating

The composition was compressed using a compressor for the production of a multi-layered tablet (MRC-37: Sejong). In detail, the composition comprising simvastatin was input in a first power inlet and the composition comprising amlodipine was input in a second inlet. The compression was performed under such a condition that the interlayer incorporation may be minimized at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing multi-layered tablets.

### Example 2

### Preparation of Amlodipine-Simvastatin Multi-Layered Tablets

### 1) Preparation of Amlodipine Controlled-Release Layer

Predetermined amounts of amlodipine and noncrystalline cellulose as shown in TABLE 2 were sieved with a 35 mesh sieve, and mixed using a double cone mixer. The mixture was introduced in a high-speed mixture, combined with the addition of Kollicoat SR30D, granulated using a oscillator with a 20 mesh sieve sieve, dried at 60° C. using a hot-water drier, and finally mixed with a magnesium stearate using a double cone mixer.

### 2) Preparation of Simvastatin Layer

Predetermined amounts of simvastatin, noncrystalline cellulose and mannitol as shown in TABLE 2 were sieved with a 35 mesh sieve and mixed using a high-speed mixer. A binder solution prepared by dissolving hydroxypropyl cellulose and citric acid in water, and combining with the mixture of the main ingredients in a high-speed mixer. Thus obtained mixture was granulated using an oscillator with a 20 mesh sieve, dried at 60° C. using a hot-water drier and ground with a 20 mesh sieve. The granules were mixed with butylhydroxyaniline, sodium starch glyconate and colloidal silicon dioxide, and finally mixed with magnesium stearate.

### 3) Compression and Coating

The composition was compressed using a compressor for the production of a multi-layered tablet (MRC-37: Sejong). In detail, the composition comprising simvastatin was input in a first power inlet and the composition comprising amlodipine was input in a second inlet. The compression was performed under such a condition that the interlayer incorporation may be minimized at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing multi-layered tablets.

### Example 6

### Preparation of Amlodipine-Simvastatin Multi-Layered Tablets

### 1) Preparation of Amlodipine Controlled-Release Layer

Predetermined amounts of amlodipine and noncrystalline cellulose as shown in TABLE 2 were sieved with a 35 mesh sieve, and mixed using a double cone mixer. The mixture was sprayed with a binder solution (an aqueous solution of hydroxypropylmethyl cellulose) to prepare granules. After the granules were dried, they were coated by spraying a 5 wt % solution of Eudragit RS PO in a 1:1 mixture of ethanol and methylene chloride. The coated granules were mixed with magnesium stearate using a double cone mixer.

### 2) Preparation of Simvastatin Layer

Predetermined amounts of simvastatin, noncrystalline cellulose and mannitol as shown in TABLE 2 were sieved with a 35 mesh sieve and mixed using a high-speed mixer. A binder solution prepared by dissolving hydroxypropyl cellulose and citric acid in water, and combining with the mixture of the main ingredients in a high-speed mixer. Thus obtained mixture was granulated using an oscillator with a 20 mesh sieve, dried at 60° C. using a hot-water drier and ground with a 20 mesh sieve. The granules were mixed with butylhydroxyanline, sodium starch glyconate and colloidal silicon dioxide, and finally mixed with magnesium stearate using a double cone mixer.

### 3) Compression and Coating

The composition was compressed using a compressor for the production of a multi-layered tablet (MRC-37: Sejong). In detail, the composition comprising simvastatin was input in a first power inlet and the composition comprising amlodipine was input in a second inlet. The compression was performed under such a condition that the interlayer incorporation may be minimized at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing multi-layered tablets.

### Example 7

### Preparation of Amlodipine-Simvastatin Inner Core Tablets

### 1) Preparation of Amlodipine Core Tablet

Predetermined amounts of amlodipine and noncrystalline cellulose as shown in TABLE 2 were sieved with a 35 mesh sieve, and mixed using a double cone mixer. The mixture was introduced into a fluidized-bed granulator (GPCG 1: Gatt) and sprayed with a binder solution (an aqueous solution of hydroxypropylmethyl cellulose) to prepare granules. After the granules were dried, they were coated by spraying a 5 wt % solution of hydroxypropylmethyl cellulose phthalate in a 1:1 mixture of ethanol and methylene chloride. The coated granules were mixed with magnesium stearate using a double cone mixer and compressed using a rotary compressor (MRC-33; Sejong) at a rate of 30 turns per minute to provide tablets with a hardness of 7-9 kp, thickness of 3.0 mm and a diameter of 5.5 mm, which was used as core tablets.

### 2) Preparation of Simvastatin Layer

Predetermined amounts of simvastatin, noncrystalline cellulose and mannitol as shown in TABLE 2 were sieved with a 35 mesh sieve and mixed using a high-speed mixer. A binder solution prepared by dissolving hydroxypropyl cellulose and citric acid in water, and combining with the mixture of the main ingredients in a high-speed mixer. Thus obtained mixture was granulated using an oscillator with a 20 mesh sieve, dried at 60° C. using a hot-water drier and ground with a 20 mesh sieve. The granules were mixed with magnesium stearate using a double cone mixer.

### 3) Compression and Coating

Compression was performed with a compressor for the production of an inner core tablet (KUD-1: Kilian) at a rate of 30 turns per minute using the amlodipine core table and the composition comprising simvastatin as an inner core and an outer layer, respectively, to provide a table with a hardness of 7-9 kp, a thickness of 6.0 mm and a diameter of 9.5 mm. After an additional compression was performed using a High-coater (SFC-30N, Sejong mechanics, Korea), a film coating layer was formed on the compressed tablets using High-coater (SFC-30N, Sejong mechanics, Korea), thus producing inner core tablets.

### Example 8

### Preparation of Amlodipine-Simvastatin Inner Core Tablets

### 1) Preparation of Amlodipine Core Tablets

Predetermined amounts of amlodipine and noncrystalline cellulose as shown in TABLE 2 were sieved with a 35 mesh sieve, and mixed using a double cone mixer. The mixture was introduced into a high-speed mixer, combined with Kollicoat SR30D and granulated using an oscillator with a 20 mesh sieve. After the granules were dried, they were ground with a 20 mesh sieve. The sized granules were mixed with magnesium stearate using a double cone mixer and compressed using a rotary compressor (MRC-33; Sejong) at a rate of 30 turns per minute to provide tablets with a hardness of 7-9 kp, thickness of 3.0 mm and a diameter of 5.5 mm, which was used as core tablets.

### 2) Preparation of Simvastatin Layer

Predetermined amounts of simvastatin, noncrystalline cellulose and mannitol as shown in TABLE 2 were sieved with a 35 mesh sieve and mixed using a high-speed mixer. A binder solution prepared by dissolving hydroxypropyl cellulose and citric acid in water, and combining with the mixture of the main ingredients in a high-speed mixer. Thus obtained mixture was granulated using an oscillator with a 20 mesh sieve, dried at 60° C. using a hot-water drier and ground with a 20 mesh sieve. The granules were mixed with magnesium stearate using a double cone mixer.

### 3) Compression and Coating

Compression was performed with a compressor for the production of an inner core tablet (KUD-1: Kilian) at a rate of 30 turns per minute using the amlodipine core table and the composition comprising simvastatin as an inner core and an outer layer, respectively, to provide a table with a hardness of 7-9 kp, a thickness of 6.0 mm and a diameter of 9.5 mm. After an additional compression was performed using a High-coater (SFC-30N, Sejong mechanics, Korea), a film coating layer was formed on the compressed tablets using High-coater (SFC-30N, Sejong mechanics, Korea), thus producing inner core tablets.

### Example 9

### Preparation of Amlodipine-Simvastatin Two-Phase Capsules

### 1) Preparation of Amlodipine Controlled-Release Granules

Predetermined amounts of amlodipine and noncrystalline cellulose as shown in TABLE 2 are sieved using a 35 mesh sieve, mixed using a double cone mixer. The mixture was introduced into a fluidized-bed granulator (GPCG 1: Glatt), granulated by spraying a binder solution (an aqueous solution of hydroxypropylmethyl cellulose) and dried. The granules were coated by spraying a 5 wt % solution prepared by dissolving hydroxypropylmethyl cellulose phthalate in a 1:1 mixture of ethanol and methylene chloride.

### 2) Preparation of Simvastatin Granules

Predetermined amounts of simvastatin, noncrystalline cellulose and mannitol as shown in TABLE 2 were sieved with a 35 mesh sieve and mixed and mixed using a high-speed mixer. A binder solution prepared by dissolving hydroxypropyl cellulose and citric acid in water was combined with a mixture of main ingredients in a high-speed mixer and granulated using an oscillator with a 20 mesh sieve. The granules were dried at 60° C. using a hot-water drier and ground with a 20 mesh sieve. The sized granules was added with butylhydroxyaniline and finally mixed using a double cone mixer.

### 3) Compression and Coating

The resulting composition prepared in the aforementioned processes 1) and 2) was mixed using a double cone mixer and added with sodium starch glyconate. The mixture was mixed using a double cone mixer, further mixed with colloidal silicon dioxide and finally mixed with magnesium stearate. The resulting mixture was introduced into a powder inlet and filled using a capsule filling machine.

### Example 10

### Preparation of Amlodipine-Simvastatin Two-Phase Capsules

### 1) Preparation of Amlodipine Controlled-Release Granules

Predetermined amounts of amlodipine and noncrystalline cellulose as shown in TABLE 2 were sieved using a 35 mesh sieve and mixed using a double cone mixer. The mixture was introduced into a fluidized-bed granulator (GPCG 1: Glatt), granulated by spraying Kollicoat SR30D and dried.

### 2) Preparation of Simvastatin Granules

Predetermined amounts of simvastatin, noncrystalline cellulose and mannitol as shown in TABLE 2 35 mesh sieve were sieved and mixed using a high-speed mixer. A binder solution prepared by dissolving hydroxypropyl cellulose and citric acid in water was combined with a mixture of main ingredients in a high-speed mixer and granulated using an oscillator with a 20 mesh sieve. The granules was dried at 60° C. using a hot-water drier and ground with a 20 mesh sieve. The sized granules were finally mixed with butylhydroxyaniline.

### 3) Compression and Coating

The resulting composition prepared in the aforementioned processes 1) and 2) was mixed using a double cone mixer and added with sodium starch glyconate. The mixture was mixed using a double cone mixer, further mixed with colloidal silicon dioxide using a double cone mixer and finally mixed with magnesium stearate. The resulting mixture was introduced into a powder inlet and filled using a capsule filling machine.

### Example 11

### Preparation of Amlodipine-Lovastatin Multi-Layered Tablets

### 1) Preparation of Amlodipine Controlled-Release Layer

Predetermined amounts of amlodipine and noncrystalline cellulose as shown in TABLE 3 were sieved with a 35 mesh sieve and mixed using a double cone mixer. The mixture was introduced into a fluidized-bed granulator (GPCG 1: Glatt), granulated by spraying a binder solution (an aqueous solution of hydroxypropylmethyl cellulose) and dried. The granules were coated by spraying a 5 wt % solution prepared by dissolving hydroxypropylmethyl cellulose phthalate in a 1:1 mixture of ethanol and methylene chloride, and finally mixed with magnesium stearate using a double cone mixer.

### 2) Preparation of Lovastatin Layer

Predetermined amounts of lovastatin, noncrystalline cellulose and mannitol as shown in TABLE 3 were sieved with a 35 mesh sieve and mixed using a high-speed mixer. A binder solution prepared by dissolving hydroxypropyl cellulose and citric acid in water was combined with a mixture of main ingredients in a high-speed mixer and granulated using an oscillator with a 20 mesh sieve. The granules were dried at 60° C. using a hot-water drier and ground with a 20 mesh sieve. The sized granules were mixed with butylhydroxyaniline, sodium starch glyconate and colloidal silicon dioxide and finally mixed with magnesium stearate using a double cone mixer.

### 3) Compression and Coating

Compression was performed using a compressor for the production of multi-layered tablet (MRC-37T: Sejong). In detail, the composition comprising simvastatin was input in a first power inlet and the composition comprising amlodipine was input in a second inlet. The compression was performed under such a condition that the interlayer incorporation may be minimized at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing multi-layered tablets.

### Example 12

### Preparation of Amlodipine-Lovastatin Multi-Layered Tablets

### 1) Preparation of Amlodipine Controlled-Release Layer

Predetermined amounts of amlodipine and noncrystalline cellulose as shown in TABLE 3 were sieved with a 35 mesh sieve and mixed using a double cone mixer. The mixture was introduced into a high-speed mixer, combined by adding Kollicoat SR30D and granulated using an oscillator with a 20 mesh sieve. The granules were dried at 60° C. using a hot-water drier and tableted with a 20 mesh sieve. The sized granules were finally mixed with magnesium stearate using a double cone mixer.

### 2) Preparation of Lovastatin Layer

Predetermined amounts of lovastatin, noncrystalline cellulose and mannitol as shown in TABLE 3 were sieved with a 35 mesh sieve and mixed using a high-speed mixer. A binder solution prepared by dissolving hydroxypropyl cellulose and citric acid in water was combined with a mixture of main ingredients in a high-speed mixer and granulated using an oscillator with a 20 mesh sieve. The granules were dried at 60° C. using a hot-water drier and ground with a 20 mesh sieve. The sized granules were mixed with butylhydroxyaniline, sodium starch glyconate and colloidal silicon dioxide and finally mixed with magnesium stearate using a double cone mixer.

### 3) Compression and Coating

Compression was performed using a compressor for the production of multi-layered tablet (MRC-37T: Sejong). In detail, the composition comprising lovastatin was input in a first power inlet and the composition comprising amlodipine was input in a second inlet. The compression was performed under such a condition that the interlayer incorporation may be minimized at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing multi-layered tablets.

### Example 13

### Preparation of Amlodipine-Atrovastatin Multi-Layered Tablets

### 1) Preparation of Amlodipine Controlled-Release Layer

Predetermined amounts of amlodipine and noncrystalline cellulose as shown in TABLE 3 were sieved with a 35 mesh sieve and mixed using a double cone mixer. The mixture was introduced into a fluidized-bed granulator (GPCG 1: Glatt), granulated by spraying a binder solution (an aqueous solution of hydroxypropylmethyl cellulose) and dried. The granules were coated by spraying a 5 wt % solution prepared by dissolving hydroxypropylmethyl cellulose phthalate in a 1:1 mixture of ethanol and methylene chloride. The coated granules were finally mixed with magnesium stearate using a double cone mixer.

### 2) Preparation of Atrovastatin Layer

Predetermined amounts of atrovastatin, noncrystalline cellulose and mannitol as shown in TABLE 3 were sieved with a 35 mesh sieve and mixed using a high-speed mixer. A binder solution prepared by dissolving hydroxypropyl cellulose and citric acid in water was combined with a mixture of main ingredients in a high-speed mixer and granulated using an oscillator with a 20 mesh sieve. The granules were dried at 60° C. using a hot-water drier and ground with a 20 mesh sieve. The sized granules were mixed with butylhydroxyaniline, sodium starch glyconate and colloidal silicon dioxide and finally mixed with magnesium stearate using a double cone mixer.

### 3) Compression and Coating

Compression was performed using a compressor for the production of multi-layered tablet (MRC-37T: Sejong). In detail, the composition comprising atrovastatin was input in a first power inlet and the composition comprising amlodipine was input in a second inlet. The compression was performed under such a condition that the interlayer incorporation may be minimized at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing multi-layered tablets.

### Example 14

### Preparation of Amlodipine-Atrovastatin Multi-Layered Tablets

### 1) Preparation of Amlodipine Controlled-Release Layer

Predetermined amounts of amlodipine and noncrystalline cellulose as shown in TABLE 3 were sieved with a 35 mesh sieve and mixed using a double cone mixer. The mixture was introduced into a high-speed mixer, combined by adding Kollicoat SR30D and granulated using an oscillator with a 20 mesh sieve. The granules were dried at 60° C. using a hot-water drier and ground with a 20 mesh sieve. The sized granules were mixed with magnesium stearate using a double cone mixer.

### 2) Preparation of Atrovastatin Layer

Predetermined amounts of atrovastatin, noncrystalline cellulose and mannitol as shown in TABLE 3 were sieved with a 35 mesh sieve and mixed using a high-speed mixer. A binder solution prepared by dissolving hydroxypropyl cellulose and citric acid in water was combined with a mixture of main ingredients in a high-speed mixer and granulated using an oscillator with a 20 mesh sieve. The granules were dried at 60° C. using a hot-water drier and ground with a 20 mesh sieve. The sized granules were mixed with butylhydroxyaniline, sodium starch glyconate and colloidal silicon dioxide and finally mixed with magnesium stearate using a double cone mixer.

### 3) Compression and Coating

Compression was performed using a compressor for the production of multi-layered tablet (MRC-37T: Sejong). In detail, the composition comprising atrovastatin was input in a first power inlet and the composition comprising amlodipine was input in a second inlet. The compression was performed under such a condition that the interlayer incorporation may be minimized at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing multi-layered controlled-release tablets

### Example 15

### Preparation of Lercanidipine-Simvastatin Multi-Layered Tablets

### 1) Preparation of Lercanidipine Controlled-Release Layer

Predetermined amounts of lercanidipine and noncrystalline cellulose as shown in TABLE 3 were sieved with a 35 mesh sieve and mixed using a double cone mixer. The mixture were introduced into a fluidized-bed granulator (GPCG 1: Glatt), granulated by spraying a binder solution (an aqueous solution of hydroxypropylmethyl cellulose) and dried. The granules were coated by spraying a 5 wt % solution prepared by dissolving hydroxypropylmethyl cellulose phthalate in a 1:1 mixture of ethanol and methylene chloride. The coated granules were finally mixed with magnesium stearate using a double cone mixer.

### 2) Preparation of Simvastatin Layer

Predetermined amounts of simvastatin, noncrystalline cellulose and mannitol as shown in TABLE 3 were sieved with a 35 mesh sieve and mixed using a high-speed mixer. A binder solution prepared by dissolving hydroxypropyl cellulose and citric acid in water was combined with a mixture of main ingredients in a high-speed mixer and granulated using an oscillator with a 20 mesh sieve. The granules were dried at 60° C. using a hot-water drier and ground with a 20 mesh sieve. The sized granules were mixed with butylhydroxyaniline, sodium starch glyconate and colloidal silicon dioxide and finally mixed with magnesium stearate using a double cone mixer.

### 3) Compression and Coating

Compression was performed using a compressor for the production of multi-layered tablet (MRC-37T: Sejong). In detail, the composition comprising simvastatin was introduced into a first powder inlet and the composition comprising lercanidipine was introduced into a second powder inlet. The compression was performed under such a condition that the interlayer incorporation may be minimized at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing multi-layered tablets.

### Example 16

### Preparation of Lercanidipine-Simvastatin Multi-Layered Tablets

### 1) Preparation of Lercanidipine Controlled-Release Layer

Predetermined amounts of lercanidipine and noncrystalline cellulose as shown in TABLE 3 were sieved with a 35 mesh sieve and mixed using a double cone mixer. The mixture was introduced into a high-speed mixer, combined by adding Kollicoat SR30D and granulated using an oscillator with a 20 mesh sieve. The granules were dried at 60° C. using a hot-water drier and tableted with a 20 mesh sieve. The sized granules were finally mixed with magnesium stearate using a double cone mixer.

### 2) Preparation of Simvastatin Layer

Predetermined amounts of simvastatin, noncrystalline cellulose and mannitol as shown in TABLE 3 were sieved with a 35 mesh sieve and mixed using a high-speed mixer. A binder solution prepared by dissolving hydroxypropyl cellulose and citric acid in water was combined with a mixture of main ingredients in a high-speed mixer and granulated using an oscillator with a 20 mesh sieve. The granules were dried at 60° C. using a hot-water drier and ground with a 20 mesh sieve. The sized granules were mixed with butylhydroxyaniline, sodium starch glyconate and colloidal silicon dioxide and finally mixed with magnesium stearate using a double cone mixer.

### 3) Compression and Coating

Compression was performed using a compressor for the production of multi-layered tablet (MRC-37T: Sejong). In detail, the composition comprising simvastatin was introduced into a first powder inlet, and then the composition comprising lercanidipine was introduced into a second powder inlet. The compression was performed under such a condition that the interlayer incorporation may be minimized at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing multi-layered tablets.

### Example 17

### Preparation of Lacidipine-Simvastatin Multi-Layered Tablets

### 1) Preparation of Lercanidipine Controlled-Release Layer

Predetermined amounts of lacidipine and noncrystalline cellulose as shown in TABLE 3 were sieved with a 35 mesh sieve and mixed using a double cone mixer. The mixture was introduced into a fluidized-bed granulator (GPCG 1: Glatt), granulated by spraying a binder solution (an aqueous solution of hydroxypropylmethyl cellulose) and dried. The granules were coated by spraying a 5 wt % solution prepared by dissolving hydroxypropylmethyl cellulose phthalate in a 1:1 mixture of ethanol and methylene chloride. The coated granules were finally mixed with magnesium stearate using a double cone mixer.

### 2) Preparation of Simvastatin Layer

Predetermined amounts of simvastatin, noncrystalline cellulose and mannitol as shown in TABLE 3 were sieved with a 35 mesh sieve and mixed using a high-speed mixer. A binder solution prepared by dissolving hydroxypropyl cellulose and citric acid in water was combined with a mixture of main ingredients in a high-speed mixer and granulated using an oscillator with a 20 mesh sieve. The granules were dried at 60° C. using a hot-water drier and ground with a 20 mesh sieve. The sized granules were mixed with butylhydroxyaniline, sodium starch glyconate and colloidal silicon dioxide, and finally mixed with magnesium stearate using a double cone mixer.

### 3) Compression and Coating

Compression was performed using a compressor for the production of multi-layered tablet (MRC-37T: Sejong). In detail, the composition comprising simvastatin was introduced into a first powder inlet, and the composition comprising lacidipine was introduced into a second powder inlet. The compression was performed under such a condition that the interlayer incorporation may be minimized at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing multi-layered tablets.

### Example 18

### Preparation of Lacidipine-Simvastatin Multi-Layered Tablets

### 1) Preparation of Lercanidipine Controlled-Release Layer

Predetermined amounts of amlodipine and noncrystalline cellulose as shown in TABLE 3 were sieved with a 35 mesh sieve and mixed combined by adding Kollicoat SR30D and granulated using an oscillator with a 20 mesh sieve. The granules were dried at 60° C. using a hot-water drier and ground with a 20 mesh sieve. The sized granules were finally mixed with magnesium stearate using a double cone mixer.

### 2) Preparation of Simvastatin Layer

Predetermined amounts of simvastatin, noncrystalline cellulose and mannitol as shown in TABLE 3 were sieved with a 35 mesh sieve and mixed using a high-speed mixer. A binder solution prepared by dissolving hydroxypropyl cellulose and citric acid in water was combined with a mixture of main ingredients in a high-speed mixer and granulated using an oscillator with a 20 mesh sieve. The granules were dried at 60° C. using a hot-water drier and ground with a 20 mesh sieve. The sized granules were mixed with butylhydroxyaniline, sodium starch glyconate and colloidal silicon dioxide, and finally mixed with magnesium stearate using a double cone mixer.

### 3) Compression and Coating

Compression was performed using a compressor for the production of multi-layered tablet (MRC-37T: Sejong). In detail, the composition comprising simvastatin was introduced into a first powder inlet, and the composition comprising lacidipine was introduced into a second powder inlet. The compression was performed under such a condition that the interlayer incorporation may be minimized at a speed of 30 turns per minute to provide tablets with a hardness of 7-9 kp, a thickness of 6.0 mm and a diameter of 9.5 mm. Film coating layer was formed on the compressed tablets using High-coater (SFC-30N, Sejong mechanics, Korea), thus producing multi-layered tablets.

TABLE 2

| | | Amounts (mg/tablet) | | | | | | | | | |
|---|---|---|---|---|---|---|---|---|---|---|---|
| | | Examples | | | | | | | | | |
| Ingredients | | 1 | 2 | 3 | 4 | 5 | 6 | 7 | 8 | 9 | 10 |
| Controlled- | Amlodipine maleate | 6.42 | 6.42 | 6.42 | 6.42 | 6.42 | 6.42 | 6.42 | 6.42 | 6.42 | 6.42 |
| release | Lercanidipine HCl | — | — | — | — | — | — | — | — | — | — |
| layer | Lacidipine | — | — | — | — | — | — | — | — | — | — |
| | Noncrystalline | 88.58 | 81.58 | 81.58 | 87.83 | 80.83 | 80.83 | 87.83 | 80.83 | 88.58 | 81.58 |
| | cellulose | | | | | | | | | | |
| | Kollicoat SR30D¹⁾ | — | 12 | — | — | 12 | — | — | 12 | — | 12 |
| | Eudragit RS PO²⁾ | — | — | 10 | — | — | 10 | — | — | — | — |
| | Hydroxypropylmethyl | 2 | — | 2 | 2 | — | 2 | 2 | — | 2 | — |
| | cellulose | | | | | | | | | | |
| | Hydroxypropylmethyl | 3 | — | — | 3 | — | — | 3 | — | 3 | — |
| | cellulose phthalate | | | | | | | | | | |
| | Magnesium stearate | — | — | — | 0.75 | 0.75 | 0.75 | 0.75 | 0.75 | — | — |
| Immediate- | Simvastatin | 20 | 20 | 20 | 20 | 20 | 20 | 20 | 20 | 20 | 20 |
| release | Lovastatin | — | — | — | — | — | — | — | — | — | — |
| layer | Atorvastatin | — | — | — | — | — | — | — | — | — | — |
| | Noncrystalline | 57 | 57 | 57 | 57 | 57 | 57 | 57 | 57 | 57 | 57 |
| | cellulose | | | | | | | | | | |
| | Di-mannitol | 112.46 | 112.46 | 112.46 | 112.46 | 112.46 | 112.46 | 112.46 | 112.46 | 112.46 | 112.46 |
| | Sodium starch | 1 | 1 | 1 | 1 | 1 | 1 | 1 | 1 | 1 | 1 |
| | glyconate | | | | | | | | | | |
| | Butylhydroxyaniline | 0.04 | 0.04 | 0.04 | 0.04 | 0.04 | 0.04 | 0.04 | 0.04 | 0.04 | 0.04 |
| | Hydroxypropylmethyl | 5 | 5 | 5 | 5 | 5 | 5 | 5 | 5 | 5 | 5 |
| | cellulose | | | | | | | | | | |
| | Aerosil 200³⁾ | 1 | 1 | 1 | 1 | 1 | 1 | 1 | 1 | 1 | 1 |
| | Citric acid | 2 | 2 | 2 | 2 | 2 | 2 | 2 | 2 | 2 | 2 |
| | Magnesium stearate | 1.5 | 1.5 | 1.5 | 1.5 | 1.5 | 1.5 | 1.5 | 1.5 | 1.5 | 1.5 |
| Coating | Hydroxymethyl | 2.6 | 2.6 | 2.6 | 2.6 | 2.6 | 2.6 | 2.6 | 2.6 | — | — |
| layer | cellulose 2910 | | | | | | | | | | |
| | Hydroxypropyl | 2.6 | 2.6 | 2.6 | 2.6 | 2.6 | 2.6 | 2.6 | 2.6 | — | — |
| | cellulose | | | | | | | | | | |
| | Titanium oxide | 2.3 | 2.3 | 2.3 | 2.3 | 2.3 | 2.3 | 2.3 | 2.3 | — | — |
| | Talc | 1.5 | 1.5 | 1.5 | 1.5 | 1.5 | 1.5 | 1.5 | 1.5 | — | — |
| | Ethanol | 64.8 | 64.8 | 64.8 | 64.8 | 64.8 | 64.8 | 64.8 | 64.8 | — | — |
| | Distilled water | 16.2 | 16.2 | 16.2 | 16.2 | 16.2 | 16.2 | 16.2 | 16.2 | — | — |

| | | | | | | | | | | |
|---|---|---|---|---|---|---|---|---|---|---|
| Total | 309 | 309 | 309 | 309 | 309 | 309 | 309 | 309 | 300 | 300 |

¹⁾Kollicoat SR30D - Main ingredient: polyacetate 30% suspension (BASF)
²⁾Eudragit RS PO - Main ingredient: polymethacrylate copolymer (BASF)
³⁾Aerosil 200 - Main ingredient: colloidal silicon dioxide (Degussa)

TABLE 3

| | | Amounts (mg/tablet) | | | | | | | |
|---|---|---|---|---|---|---|---|---|---|
| | | Examples | | | | | | | |
| Ingredients | | 11 | 12 | 13 | 14 | 15 | 16 | 17 | 18 |
| Controlled- | Amlodipine maleate | 6.42 | 6.42 | 6.42 | 6.42 | — | — | — | — |
| release | Lercanidipine HCl | — | — | — | — | 10- | 10 | — | — |
| layer | Lacidipine | — | — | — | — | — | — | 4 | 4 |
| | Noncrystalline | 87.83 | 80.83 | 87.83 | 80.83 | 79.25 | 74.25 | 90.25 | 83.25 |
| | cellulose | | | | | | | | |
| | Kollicoat SR30D¹⁾ | — | 12 | — | 12 | — | 15 | — | 12 |
| | Eudragit RS PO²⁾ | — | — | — | — | — | — | — | — |
| | Hydroxypropylmethyl | 2 | — | 2 | — | 4 | — | 2 | — |
| | cellulose | | | | | | | | |
| | Hydroxypropylmethyl | 3 | — | 3 | — | 6 | — | 3 | — |
| | cellulose phthalate | | | | | | | | |
| | Magnesium stearate | 0.75 | 0.75 | 0.75 | 0.75 | 0.75 | 0.75 | 0.75 | 0.75 |
| Immediate- | Simvastatin | — | — | — | — | 20 | 20 | 20 | 20 |
| release | Lovastatin | 20 | 20 | — | — | — | — | — | — |
| layer | Atorvastatin | — | — | 20 | 20 | — | — | — | — |
| | Noncrystalline | 57 | 57 | 57 | 57 | 57 | 57 | 57 | 57 |
| | cellulose | | | | | | | | |
| | Di-mannitol | 112.46 | 112.46 | 112.46 | 112.46 | 112.46 | 112.46 | 112.46 | 112.46 |
| | Sodium starch | 1 | 1 | 1 | 1 | 1 | 1 | 1 | 1 |
| | glyconate | | | | | | | | |
| | Butylhydroxyaniline | 0.04 | 0.04 | 0.04 | 0.04 | 0.04 | 0.04 | 0.04 | 0.04 |
| | Hydroxypropylmethyl | 5 | 5 | 5 | 5 | 5 | 5 | 5 | 5 |
| | cellulose | | | | | | | | |
| | Aerosil 200³⁾ | 1 | 1 | 1 | 1 | 1 | 1 | 1 | 1 |
| | Citric acid | 2 | 2 | 2 | 2 | 2 | 2 | 2 | 2 |
| | Magnesium stearate | 1.5 | 1.5 | 1.5 | 1.5 | 1.5 | 1.5 | 1.5 | 1.5 |
| Coating | Hydroxymethyl | 2.6 | 2.6 | 2.6 | 2.6 | 2.6 | 2.6 | 2.6 | 2.6 |
| layer | cellulose 2910 | | | | | | | | |
| | Hydroxypropyl | 2.6 | 2.6 | 2.6 | 2.6 | 2.6 | 2.6 | 2.6 | 2.6 |
| | cellulose | | | | | | | | |
| | Titanium oxide | 2.3 | 2.3 | 2.3 | 2.3 | 2.3 | 2.3 | 2.3 | 2.3 |
| | Talc | 1.5 | 1.5 | 1.5 | 1.5 | 1.5 | 1.5 | 1.5 | 1.5 |
| | Ethanol | 64.8 | 64.8 | 64.8 | 64.8 | 64.8 | 64.8 | 64.8 | 64.8 |
| | Distilled water | 16.2 | 16.2 | 16.2 | 16.2 | 16.2 | 16.2 | 16.2 | 16.2 |

| | | | | | | | | |
|---|---|---|---|---|---|---|---|---|
| Total | 309 | 309 | 309 | 309 | 309 | 309 | 309 | 309 |

¹⁾Kollicoat SR30D - Main ingredient: polyacetate 30% suspension (BASF)
²⁾Eudragit RS PO - Main ingredient: polymethacrylate copolymer (BASF)
³⁾Aerosil 200 - Main ingredient: colloidal silicon dioxide (Degussa)

### Experimental Example 1

### Comparative Dissolution Profile Test

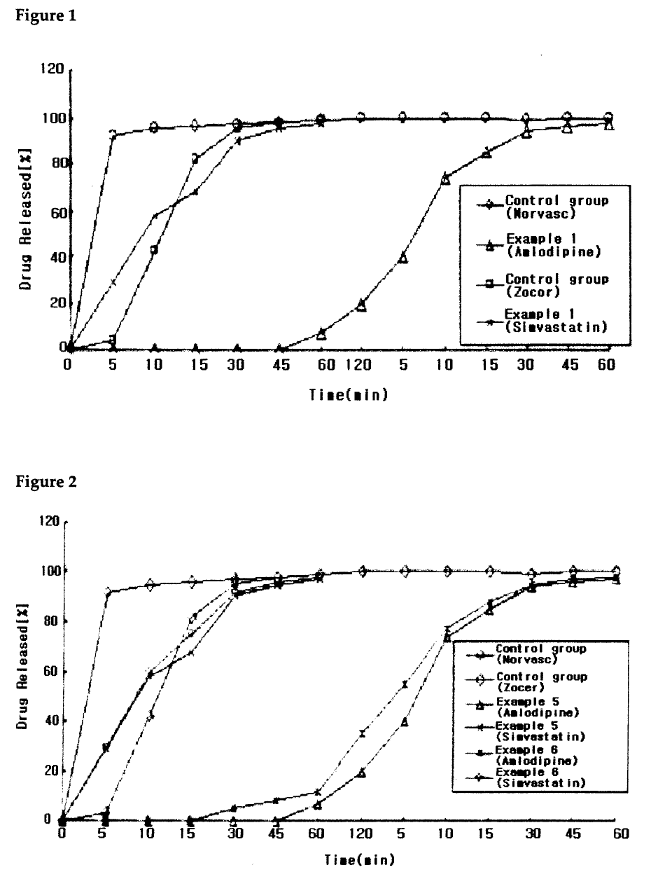

Comparative dissolution profile test was performed using an amlodipine/simvastatin two-phase matrix tablets prepared in Example 1 and control drugs (Zocor: simvastatin single pill, Norvasc: amlodipine single pill). In the case of dissolution profile test of amlodipine ingredient, the dissolution solution was changed from an artificial gastric juice to an artificial intestinal juice after 2 hours. Specific process of dissolution profile test of each ingredient is described below, and the results are presented in FIG. 1.

When the dissolution profile test was performed under the conditions described below, in the two-phase matrix tablets according to the present invention, the simvastatin ingredient showed a nearly equivalent dissolution behavior as compared to that of the control drug (Zocor), while the amlodipine ingredient showed a very retarded dissolution rate as compared to that of the control drug (Norvasc). In the case of the amlodipine/simvastatin two-phase matrix tablet according to the present invention, dissolution rates of amlodipine ingredient were all within 50% until one hour after the test began, which were far lower than that of the control drug (about 99%).

As described above, in the amlodipine/simvastatin two-phase matrix tablet according to the present invention, amlodipine shows a far lower initial dissolution rate than simvastatin unlike the control drugs (i.e. amlodipine single pill), and thus the amlodipine/simvastatin two-phase matrix tablet according to the present invention is less likely to be subject to the metabolism in liver ahead of simvastatin.

### Test Method for Amlodipine

Based on the general dissolution test method described in Korea Pharmacopoeia (8th revision)

Test method: Paddle method), 75 turns/minute

Dissolution solution: 0.01 M chloric acid solution, 750 mL

Analysis method: UV-visible spectrophotometry (detected wavelength maximum 240 nm)

### Test Method for Simvastatin

Based on the ‘Simvastatin tablet’ part in USFDA (USP 29)

Test method: Paddle method, 50 turns/minute

Dissolution solution: pH=7.0 buffer solution (0.01 M monobasic sodium phosphate solution containing sodium lauryl sulfate 0.5% wt/wt as surfactant), 900 mL

Analysis method: UV-visible spectrophotometry (detected wavelength=maximum 247 nm and minimum 257 nm)

### Experimental Example 2

### Comparative Dissolution Profile Test

Comparative dissolution profile test was performed using an amlodipine/simvastatin combined pharmaceutical formulation prepared in Examples 5 and 6 and control drugs (Zocor: simvastatin single pill, Norvasc: amlodipine single pill). The dissolution behavior of simvastatin and amlodipine was observed as described below, and in the case of dissolution profile test of amlodipine ingredient, the dissolution solution was changed from an artificial gastric juice to an artificial intestinal juice after 2 hours. Specific process of dissolution profile test of each ingredient is described below, and the results are presented in FIG. 2.

When the dissolution profile test was performed under the conditions described below in Examples 5 and 6, the simvastatin ingredient showed a nearly equivalent dissolution behavior as compared to that of the control drug (Zocor), while the amlodipine ingredient showed a very retarded dissolution rate as compared to that of the control drug (Norvasc). In the case of the amlodipine/simvastatin multi-layered tablet according to the present invention, dissolution rates of amlodipine ingredient were all within 50% until one hour after the test began, which were far lower than that of the control drug (about 99%).

As described above, in the amlodipine/simvastatin multi-layered tablet according to the present invention, amlodipine shows a far lower initial dissolution rate than simvastatin unlike the control drugs (i.e. amlodipine single pill), and thus the amlodipine/simvastatin multi-layered tablet according to the present invention is less likely to be subject to the metabolism in liver ahead of simvastatin.

### Test Method for Amlodipine

Based on the general dissolution test method described in Korea Pharmacopoeia (8th revision)

Test method: Paddle method), 75 turns/minute

Dissolution solution: 0.01 M chloric acid solution, 750 mL

Analysis method: UV-visible spectrophotometry (detected wavelength=maximum 240 nm)

### Test Method for Simvastatin

Based on the ‘Simvastatin tablet’ part in USFDA (USP 29)

Test method: Paddle method, 50 turns/minute

Dissolution solution: pH=7.0 buffer solution (0.01 M monobasic sodium phosphate solution containing sodium lauryl sulfate 0.5% wt/wt as surfactant), 900 mL

Analysis method: UV-visible spectrophotometry (detected wavelength=maximum 247 nm and minimum 257 nm)

### Experimental Example 3

### Comparative Dissolution Profile Test

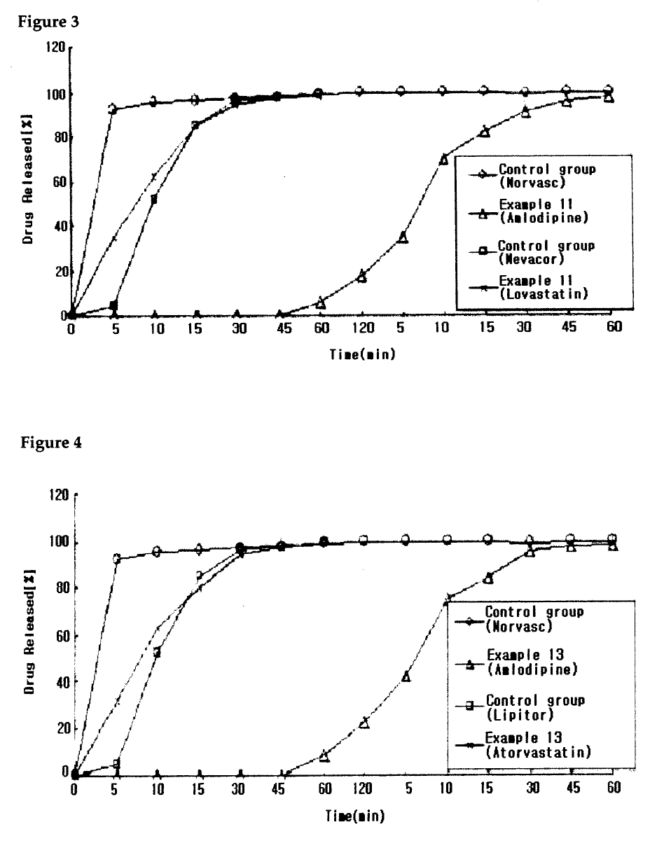

Comparative dissolution profile test was performed using an amlodipine/lovastatin combined pharmaceutical formulation prepared in Example 11 and control drugs (Mevacor: lovastatin single pill, Norvasc: amlodipine single pill). In the case of dissolution profile test of amlodipine ingredient, the dissolution solution was changed from an artificial gastric juice to an artificial intestinal juice after 2 hours. Specific process of dissolution profile test of each ingredient is described below, and the results are presented in FIG. 3.

When the dissolution profile test was performed under the conditions described below in Example 11, lovastatin ingredient showed a nearly equivalent dissolution behavior as compared to that of the control drug (Mevacor), while amlodipine ingredient showed a very retarded dissolution rate as compared to that of the control drug (Norvasc). In the case of the amlodipine/lovastatin multi-layered tablet according to the present invention, dissolution rates of amlodipine ingredient were all within 50% until one hour after the test began, which were far lower than that of the control drug (about 99%).

As described above, in the amlodipine/lovastatin multi-layered tablet according to the present invention, amlodipine shows a far lower initial dissolution rate than that of lovastatin unlike the control drugs (i.e. amlodipine single pill), and thus the amlodipine/lovastatin multi-layered tablet according to the present invention is less likely to be subject to the metabolism in liver ahead of lovastatin.

### Test Method for Amlodipine

Based on the general dissolution test method described in Korea Pharmacopoeia (8th revision)

Test method: Paddle method), 75 turns/minute

Dissolution solution: 0.01 M chloric acid solution, 750 mL

Analysis method: UV-visible spectrophotometry (detected wavelength=maximum 240 nm)

### Test Method for Lovastatin

Based on the ‘Lovastatin tablet’ part in USFDA (USP 29)

Test method: Paddle method, 50 turns/minute

Dissolution solution: pH=7.0 buffer solution (0.01 M monobasic sodium phosphate solution containing sodium lauryl sulfate 0.5% wt/wt as surfactant), 900 mL

Analysis method: High performance liquid chromatography

Detected wavelength: 230 nm

Mobile phase: Acetronitrile: 0.02 M monobasic sodium phosphate buffer solution (pH=4.0): methanol=5:3:1

Column: Octadecyl silyl silica gel packed in a stainless steel tube of 4.6 mm (internal diameter) and 250 mm (length)

Flow rate: 1.5 mL/minute

### Experimental Example 4

### Comparative Dissolution Profile Test

Comparative dissolution profile test was performed using an amlodipine/atrovastatin combined pharmeceutical formulation prepared in Example 13 and control drugs (Lipitor: atrovastatin single pill, Norvasc: amlodipine single pill). In the case of dissolution profile test of amlodipine ingredient, the dissolution solution was changed from an artificial gastric juice to an artificial intestinal juice after 2 hours. Specific process of dissolution profile test of each ingredient is described below, and the results are presented in FIG. 4.

When the dissolution profile test was performed under the conditions described below in Example 13, atrovastatin ingredient showed a nearly equivalent dissolution behavior as compared to that of the control drug (Lipitor), while amlodipine ingredient showed a very retarded dissolution rate as compared to that of the control drug (Norvasc). In the case of the amlodipine/atrovastatin multi-layered tablet according to the present invention, dissolution rates of amlodipine ingredient were all within 50% until one hour after the test began, which were far lower than that of the control drug (about 99%).

As described above, in the amlodipine/atrovastatin multi-layered tablet according to the present invention, amlodipine shows a far lower initial dissolution rate than atrovastatin unlike the control drugs (i.e. amlodipine single pill), and thus the amlodipine/atrovastatin multi-layered tablet according to the present invention is less likely to be subject to the metabolism in liver ahead of atrovastatin.

### Test Method for Amlodipine

Based on the general dissolution test method described in Korea Pharmacopoeia (8th revision)

Test method: Paddle method), 75 turns/minute

Dissolution solution: 0.01 M chloric acid solution, 750 mL

Analysis method: UV-visible spectrophotometry (detected wavelength maximum 240 nm)

### Test Method for Atrovastatin

Based on the general dissolution test method described in Korea Pharmacopoeia (8th revision)

Test method: Paddle method, 50 turns/minute

Dissolution solution: pH=7.0 buffer solution (0.01 M monobasic sodium phosphate solution containing sodium lauryl sulfate 2% wt/wt as surfactant), 900 mL

Analysis method: High performance liquid chromatography

Detected wavelength: 247 nm

Mobile phase: Methanol: 0.025 M monobasic sodium phosphate buffer solution (pH=4.0): methanol=67:33 (pH=4.0)

Column: Octadecyl silyl silica gel packed in a stainless steel tube of 4.6 mm (internal diameter) and 250 mm (length)

Flow rate: 1.5 mL/minute

### Experimental Example 5

### Comparative Dissolution Profile Test

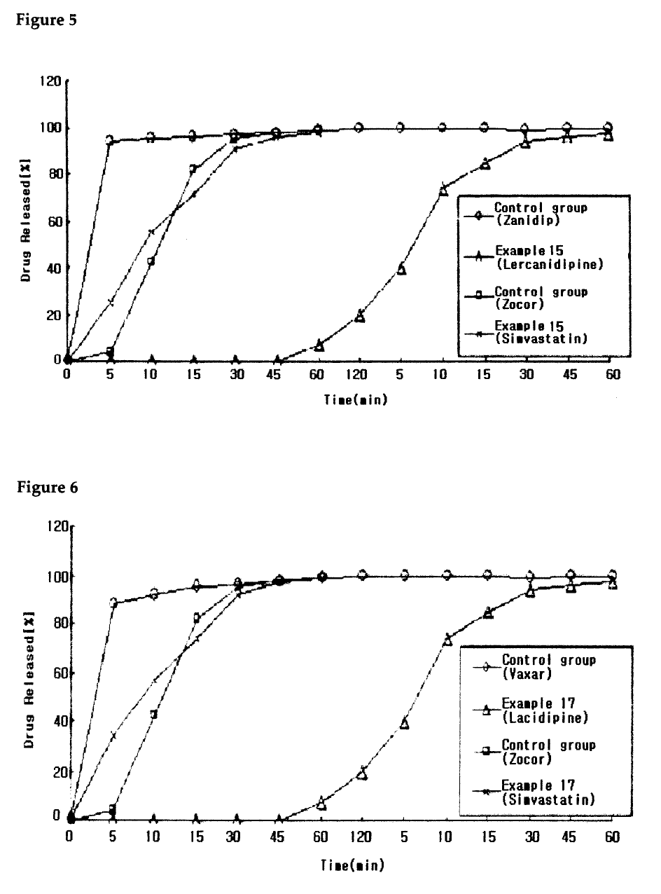

Comparative dissolution profile test was performed using a lercanidipine/simvastatin combined pharmeceutical formulation prepared in Example 15 and control drugs (Zocor: simvastatin single pill, Zanidip: lercanidipine single pill). In the case of dissolution profile test of lercanidipine ingredient, the dissolution solution was changed from an artificial gastric juice to an artificial intestinal juice after 2 hours. Specific process of dissolution profile test of each ingredient is described below, and the results are presented in FIG. 5.

When the dissolution profile test was performed under the conditions described below in Example 15, the simvastatin ingredient showed a nearly equivalent dissolution behavior as compared to that of the control drug (Zocor), while the lercanidipine ingredient showed a very retarded dissolution rate as compared to that of the control drug (Zanidip). In the case of the lercanidipine/simvastatin multi-layered tablet according to the present invention, dissolution rates of lercanidipine ingredient were all within 50% until one hour after the test began, which were far lower than that of the control drug (about 99%).

As described above, in the lercanidipine/simvastatin multi-layered tablet according to the present invention, lercanidipine shows a far lower initial dissolution rate than simvastatin unlike the control drugs (i.e. lercanidipine single pill), and thus the lercanidipine/simvastatin multi-layered tablet according to the present invention is less likely to be subject to the metabolism in liver ahead of simvastatin.

### Test Method for Lercanidipine

Based on the general dissolution test method described in Korea Pharmacopoeia (8th revision)

Test method: Paddle method), 75 turns/minute

Dissolution solution: 0.01 M chloric acid solution, 750 mL

Analysis method: High performance liquid chromatography

Detected wavelength: 356 nm

Mobile phase: Acetronitrile: 0.01 M sodium phosphate buffer solution=45: 55 (pH=4.0)

Column: Octadecyl silyl silica gel packed in a stainless steel tube of 4.6 mm (internal diameter) and 250 mm (length)

Flow rate: 1.0 mL/minute

### Test Method for Simvastatin

Based on the ‘Simvastatin tablet’ part in USFDA (USP 29)

Test method: Paddle method, 50 turns/minute

Dissolution solution: pH=7.0 buffer solution (0.01 M monobasic sodium phosphate solution containing sodium lauryl sulfate 0.5% wt/wt as surfactant), 900 mL

Analysis method: UV-visible spectrophotometry (detected wavelength maximum 247 nm and minimum 257 nm)

### Experimental Example 6

### Comparative Dissolution Profile Test

Comparative dissolution profile test was performed using a lacidipine/simvastatin combined pharmeceutical formulation prepared in Example 17 and control drugs (Zocor: simvastatin single pill, Vaxar: lacidipine single pill). In the case of dissolution profile test of lacidipine ingredient, the dissolution solution was changed from an artificial gastric juice to an artificial intestinal juice after 2 hours. Specific process of dissolution profile test of each ingredient is described below, and the results are presented in FIG. 6.

When the dissolution profile test was performed under the conditions described below in Example 15, the simvastatin ingredient showed a nearly equivalent dissolution behavior as compared to that of the control drug (Zocor), while the lacidipine ingredient showed a very retarded dissolution rate as compared to that of the control drug (Vaxar). In the case of the lacidipine/simvastatin multi-layered tablet according to the present invention, dissolution rates of lacidipine ingredient were all within 50% until one hour after the test began, which were far lower than that of the control drug (about 99%).

As described above, in the lacidipine/simvastatin multi-layered tablet according to the present invention, lacidipine shows a far lower initial dissolution rate than simvastatin unlike the control drugs (i.e. lacidipine single pill), and thus the lacidipine/simvastatin multi-layered tablet according to the present invention is less likely to be subject to the metabolism in liver ahead of simvastatin.

### Test Method for Lacidipine

Based on the general dissolution test method described in Korea Pharmacopoeia (8th revision)

Test method: Paddle method), 75 turns/minute

Dissolution solution: 0.01 M chloric acid solution, 750 mL

Analysis method: High performance liquid chromatography

Detected wavelength: 282 nm

Mobile phase: Acetronitrile: 0.05 M ammonium acetate buffer solution=80:20

Column: Octadecyl silyl silica gel packed in a stainless steel tube of 4.6 mm (internal diameter) and 250 mm (length)

Flow rate: 1.0 mL/minute

### Test Method for Simvastatin

Based on the ‘Simvastatin tablet’ part in USFDA (USP 29)

Test method: Paddle method, 50 turns/minute

Dissolution solution: pH=7.0 buffer solution (0.01 M monobasic sodium phosphate solution containing sodium lauryl sulfate 0.5% wt/wt as surfactant), 900 mL

Analysis method: UV-visible spectrophotometry (detected wavelength=maximum 247 nm and minimum 257 nm)

As motioned above, the present invention realizes Chronotherapeutics into a formulation by pharmcokinetically improving the side effects due to the combined prescription of different drugs on a basis of Xenobiotics, thus maximizing the therapeutical effect.

The formulation of the present invention comprises statin-based lipid-lowering agent and dihydropyridine-based calcium channel blocker that affects cytochrome P450 enzyme as active ingredients, and is constituted so that the release rates of the aforementioned ingredients are different and the activities of the drugs are expressed at certain intervals.

As a result, the formulation of the present invention is more useful pharmacologically, clinically, scientifically and economically in the treatment of a chronical circulatory disorder than a combined prescription where drugs are separatedly administered at the same time.

Moreover, the combined pharmeceutical formulation of the present invention causes the drugs to be released at different rates, and prevents the antagonism and side effects, while maintaining the synergistic effect of the drugs.

Furthermore, the combined pharmaceutical formulation of the present invention is administered with a single dose, and has an advantage of convenience in medication and medication instruction.

1. A combined pharmeceutical formulation comprising a dihydropyridine-based calcium channel blocker and a statin-based lipid-lowering agent as active ingredients and a pharmaceutically acceptable carrier, the combined pharmeceutical formulation including a controlled-release part comprising the dihydropyridine-based calcium channel blocker as an active ingredient and an immediate-release part comprising the statin-based lipid-lowering agent as an active ingredient.

2. The combined pharmeceutical formulation of claim 1, wherein the dihydropyridine-based calcium channel blocker is inhibited by cytochrome P450 enzymes.

3. The combined pharmaceutical formulation of claim 1, wherein the dihydropyridine-based calcium channel blocker is selected from the group consisting of amlodipine, lercanidipine, lacidipine and a pharmaceutically acceptable salt thereof.

4. The combined pharmaceutical formulation of claim 1, wherein the dihydropyridine-based calcium channel blocker is amlodipine and a pharmaceutically acceptable salt thereof or an isomer thereof.

5. The combined pharmeceutical formulation of claim 1, wherein the dihydropyridine-based calcium channel blocker is amlodipine maleate or amlodipine besylate.

6. The combined pharmeceutical formulation of claim 1, wherein the formulation comprises 1-20 mg of dihydropyridine-based calcium channel blocker.

7. The combined pharmeceutical formulation of claim 1, wherein the controlled-release part comprises a release controlling material selected from the group consisting of an enteric polymer, a water-insoluble polymer, a hydrophobic compound, a hydrophilic non-polymeric compound and a hydrophilic polymer.

8. The combined pharmaceutical formulation of claim 7, wherein the release controlling material in the controlled-release part is contained in an amount of 10-500 weight parts relative to 100 weight parts of the dihydropyridine-based calcium channel blocker.

9. The combined pharmaceutical formulation of claim 7, wherein the enteric polymer is selected from the group consisting of poly(vinylacetate phthalate-co-methacrylic acid) copolymer, hydroxypropylmethyl cellulose phthalate, shellac, cellulose acetate phthalate, cellulose propionate phthalate, Eudragit L, Eudragit S and a mixture thereof.

10. The combined pharmeceutical formulation of claim 7, wherein the water-insoluble polymer is poly(vinylacetate-co-methacrylate) copolymer selected from the group consisting of poly(ethylacrylate-co-methylmethacrylate) copolymer, poly(ethylacrylate-methyl methacrylate-trimethyl aminoethyl methacrylate) copolymer, ethyl cellulose, cellulose acetate and a mixture thereof.

11. The combined pharmeceutical formulation of claim 7, wherein the hydrophobic organic compound is selected from the group consisting of a fatty acid and a fatty acid ester, a fatty acid alcohol, a wax, an inorganic material and a mixture thereof.

12. The combined pharmeceutical formulation of claim 11, wherein the fatty acid and fatty acid esters are selected from the group consisting of glyceryl palmitostearate, glyceryl stearate, glyceryl behenate, cetyl palmitate, glyceryl mono oleate, stearic acid and a mixture thereof; the fatty acid alcohol is selected from the group consisting of cetostearyl alcohol, cetyl alcohol, stearyl alcohol and a mixture thereof; the wax is selected from the group consisting of Carnauba wax, beeswax, noncrystalline wax and a mixture thereof; the inorganic material is selected from the group consisting of talc, precipitated calcium carbonate, dibasic calcium phosphate, zinc oxide, titanium oxide, kaolin, bentonite, montmorillonite, veegum and a mixture thereof.

13. The combined pharmeceutical formulation of claim 7, wherein the hydrophilic polymer is selected from the group consisting of a saccharide, a cellulose derivative, a gum, a protein, a polyvinyl derivative, a polymethacrylate copolymer, a polyethylene derivative, a carboxyvinyl polymer and a mixture thereof.

14. The combined pharmaceutical formulation of claim 13, wherein the saccharide is selected from the group consisting of dextrin, polydextrin, dextran, pectin and pectin derivative, alginate, poly(galacturonic acid), xylan, arabinoxylan, arabinogalactan, starch, hydroxypropyl starch, amylase, amylopectin and a mixture thereof; the cellulose derivative is selected from the group consisting of hydroxypropylmethyl cellulose, hydroxypropyl cellulose, hydroxymethyl cellulose, hydroxyethyl cellulose, methyl cellulose, carboxymethyl cellulose sodium, hydroxypropyl methyl cellulose acetate succinate, hydroxyethylmethyl cellulose and a mixture thereof; the gum is selected from the group consisting of guar gum, locust bean gum, tragacantha, carrageenan, gum acasia, gum arabic, gellan gum, xanthan gum and a mixture thereof; the protein is selected from the group consisting of gelatin, casein, zein and a mixture thereof; the polyvinyl derivative is selected from the group consisting of polyvinyl alcohol, polyvinyl pyrrolidone, poly(vinylacetal diethylaminoacetate) and a mixture thereof; the polymethacrylate copolymer is selected from the group consisting of a poly(butyl methacrylate-(2-dimethylaminoethyl) methacrylate-methyl methacrylate) copolymer, a poly(methacrylic acid-methyl methacrylate) copolymer, a poly(methacrylic acid-ethyl acrylate) copolymer and a mixture thereof; and the polyethylene derivative is selected from the group consisting of polyethylene glycol, polyethylene oxide and a mixture thereof; the carboxyvinyl polymer is carbomer.

15. The combined pharmeceutical formulation of claim 1, wherein the statin-based lipid-lowering agent is selected from the group consisting of simvastatin, lovastatin, atrovastatin and a mixture thereof.

16. The combined pharmeceutical formulation of claim 1, wherein the statin-based lipid-lowering agent is simvastatin.

17. The combined pharmeceutical formulation of claim 1, wherein the statin-based lipid-lowering agent is contained in an amount of 5-80 mg.

18. The combined pharmeceutical formulation of claim 1, which is a single pill with a two-phase matrix structure, wherein the controlled-release part is placed discontinuously, thereby causing the controlled-release of the dihydropyridine-based calcium channel blocker, and the immediate-release part is placed continuously, thereby causing the immediate-release of the statin-based lipid-lowering agent.

19. The combined pharmeceutical formulation of claim 1, wherein the controlled-release part and the immediate-release part form a multi-layered structure.

20. The combined pharmeceutical formulation of claim 1, which is a single pill with a double-layered structure comprising an inner core of the controlled-release part and an outer layer of the immediate-release part, which encompasses the inner core.

21. The combined pharmeceutical formulation of claim 1, which is a capsule comprising a granule of the controlled-release part and a granule of the immediate-release part.

22. The combined pharmeceutical formulation of claim 1, which is an uncoated tablet or a coated tablet.

23. The combined pharmeceutical formulation of claim 22, wherein the coated tablet comprise a coating layer of a film former, a film-forming adjuvant or a mixture thereof.

24. The combined pharmaceutical formulation of claim 23, wherein the coating layer comprises at least one selected from the group consisting of cellulose derivative, saccharide derivative, polyvinyl derivative, wax, fat, gelatin, polyethylene glycol, ethyl cellulose, titanium oxide, diethyl phthalate and a mixture thereof.

25. The combined pharmeceutical formulation of claim 23, wherein the coating layer is contained in an amount of 0.5-15 wt % of the total weight of the coated tablet.
